(19) United States
(12) Patent Application Publication
(10) Pub. No.: US 2026/0013878 A1
(43) Pub. Date: Jan. 15, 2026

(54) POSITIONING DEVICE

(71) Applicant: I.T.S. GmbH, Lassnitzhöhe (AT)

(72) Inventors: Ronald PRAGER, Bovenau (DE); Volker BÜHREN, Murnau (DE)

(73) Assignee: I.T.S. GmbH, Lassnitzhöhe (AT)

(21) Appl. No.: 19/109,011

(22) PCT Filed: Oct. 24, 2022
(86) PCT No.: PCT/AT2022/060367

§ 371 Date: Mar. 5, 2025

(51) Int. Cl. A61B 17/17; A61B 17/00; A61B 17/72; A61B 17/74; A61B 17/90

(52) U.S. Cl. CPC A61B 17/1717; A61B 17/1703; A61B 17/725; A61B 17/74; A61B 17/90; A61B 2017/00199

(57) ABSTRACT

Positioning device and method for positioning and/or fixing a bone implant, e.g., an intramedullary nail. The positioning device has a curved handle, having a target guide defining a target axis for guiding a bone drill device, a connection element for releasably connecting the bone implant to the curved handle and an alignment sight. The alignment sight has a first indicator for indicating the course of the target axis in a second X-ray image of the alignment sight along a target line of sight, which lies in a first alignment plane formed by the target axis and the first indicator structure. To provide an optimized handling, the alignment sight has a second indicator structure for indicating an orthogonal tilting of the line of sight from the first alignment plane in a first X-ray image of the alignment sight along a line of sight which deviates from the target line of sight.

The invention relates to a positioning device for a bone implant, in particular an intramedullary nail, for positioning and/or fixing the bone implant, typically for treating a bone fracture, in particular a fracture of a proximal femur, wherein the positioning device has a curved handle with a target guide, such as a target aperture, for guiding a bone drill device, wherein the target guide defines a target axis, and wherein the curved handle has a connection element for releasably connecting the bone implant to the curved handle, and an alignment sight, wherein the alignment sight has a first indicator structure for indicating—by means of the indicator structure—the path of the target axis in a second X-ray image of the alignment sight along a target line of sight that lies in a first alignment plane formed by the target axis and the first indicator structure.

The invention further relates to a method for treating a bone fracture, in particular a proximal femoral fracture, wherein a positioning device is used to insert an intramedullary nail into an intramedullary canal of a bone, in particular a proximal femur, wherein the intramedullary nail is connected, in particular releasably, to a curved handle of the positioning device, wherein the curved handle comprises a target guide for guiding a bone drill device into the bone along a target axis. wherein the curved handle has an alignment sight for aligning the target axis, wherein the alignment sight comprises a first indicator structure, adapted to indicate the path of the target axis in a second X-ray image of the alignment sight along a target line of sight that lies in a first alignment plane formed by the target axis and the first indicator structure.

Devices and methods of the type mentioned above for treating a fracture of a proximal femur or a femoral neck fracture are known from the state of the art. In order to treat a femoral neck fracture.

an intramedullary nail is usually introduced into the intramedullary canal of the fractured proximal femur and anchored in the bone material of the femur with a coupling device, usually a bone screw. For this purpose, the intramedullary nail often has a bore extending transversely to its longitudinal axis, through which the bone screw is introduced into the bore in a protruding manner such that one end of the bone screw projects into the femoral head. Inserting the bone screw generally requires a bone bore canal in the femur, which leads through the bore of the intramedullary nail to the femoral head. For this purpose, a guide wire is usually first introduced into the femoral head in order to then guide a cannulated drill along the guide wire in order to form the bone bore canal.

The intramedullary nail is generally introduced and positioned with a positioning device, which comprises a curved handle to which the intramedullary nail is coupled, after which a surgeon inserts and aligns the intramedullary nail into the intramedullary canal of the femur by guiding the curved handle into the intramedullary canal of the femur. The curved handle generally has a target guide for guiding the guide wire along a target axis specified by the target guide. The target axis usually extends through the bore of the intramedullary nail starting from the target guide.

A challenge for correct positioning is usually that the intramedullary nail must be correctly positioned in the femur with the positioning device and that the guide wire must be guided into a suitable depth of the femoral head. For this purpose. X-ray images are generally prepared to monitor or identify a position of the intramedullary nail and the bone drill device. In order to estimate a position of the guide wire in the region of the femoral head in relation to the femoral head, it is known to equip the curved handle with an alignment sight, wherein the alignment sight comprises an indicator that can be seen in the X-ray image, often in the shape of a U-shaped depression. The X-ray image can be used to indicate a path of the target axis when a line of sight of the X-ray image along which the X-ray image is taken lies in a first alignment plane formed by the target axis and the indicator. It is therefore necessary to align the line of sight of the X-ray image such that the line of sight, the indicator, and the target axis are located in a plane in order to estimate a position or a path of the target axis in the X-ray image and corresponding to the guide wire introduced along the target axis relative to the femoral head.

A corresponding adjustment for aligning the line of sight, indicator, and target axis has regularly proven to be a difficult task, in particular in view of a generally not particularly pronounced resolution capacity or imaging capability of the X-ray, and is associated with an extended treatment time, a pronounced radiation exposure of the patient or the surgical staff. Since the path of the target axis is generally not visible in the X-ray image, it is also often necessary to approach the femoral head with the guide wire along the target axis until the guide wire is visible in the X-ray image in order to estimate the path of the target axis on the basis of the guide wire, thus allowing the target axis, the indicator, and the line of sight of the X-ray image to be aligned with one another. Any resulting misalignment of the guide wire in the bone can cause damage to a bone material of the femur.

This is where the invention comes into play. The object of the invention is to specify a device of the aforementioned type that permits optimized treatment when implanting a bone implant. A corresponding method is also specified.

According to the invention, the first object is achieved by a device of the aforementioned type if the alignment sight comprises a second indicator structure adapted to display—in a first X-ray image—an orthogonal tilting of the line of sight from the first alignment plane along a line of sight that deviates from the target line of sight, in order to permit an alignment of the line of sight to the target line of sight.

The basis of the invention is the idea of designing the alignment sight such that a tilting of a line of sight of an X-ray image of the alignment sight in relation to the first alignment plane defined by the target axis and the first indicator structure is easily discernible in the X-ray image without error.

This is achievable if a display of an orthogonal tilting of the line of sight of the first X-ray image in relation to the first alignment plane is decoupled from the first indicator structure, especially since the first indicator structure usually requires a comparison with a path of the target axis—for example by inserting the bone drill device along the target axis—in order to detect an orthogonal tilting.

Such a misalignment can be quickly and efficiently identified by providing that the alignment sight comprises a second indicator structure, the function of which is to indicate in the first X-ray image of the alignment sight that the line of sight has an orthogonal tilting from the first alignment plane or to make such a tilting evident. The line of sight can be aligned such that it lies in the first alignment plane. The alignment sight, in particular the first indicator structure or second indicator structure, thus represents a reference point for a direction or real position of the target axis in the bone, or in particular according to the bone drill device introduced along the target axis. In particular, it is not necessary to insert the bone drill device into the bone in order to use it for an adjustment to detect the target axis. This allows the adjustment or alignment of the target axis, first indicator structure, and line of sight of the first X-ray image to be carried out simply and efficiently. so that a time requirement and/or an X-ray radiation exposure can be reduced. A potential for damage can be reduced by first performing the alignment and only thereafter having to guide the bone drill device in or into the bone. This permits an optimized treatment when implanting the bone implant.

The bone drill device can be a bone drill or a targeting wire, for example a K-wire, also called a Kirschner wire. As a rule, the invention provides that a targeting wire, in particular a K-wire, is first guided into the bone in order to define a drilling path with the targeting wire, along which a bone drill is then guided along the targeting wire. For this purpose, the bone drill usually comprises a feed-through canal for feeding through the targeting wire in order to guide the bone drill along the targeting wire. If the targeting wire is used as a guide, it is also referred to as a guide wire. The target guide defines the target axis along which the bone drill device can be guided with the target guide. The bone can be a bone part, in particular a proximal femur, or the bone fracture can be a fracture of the femur, in particular of the proximal femur.

The first or second X-ray image is generally prepared with an X-ray apparatus, which is preferably adapted such that an imaging direction or line of sight of the X-ray image from which the X-ray image is performed, can be variable, in particular controlled. It is expedient if a tilt angle of the imaging direction or line of sight is variable, in particular controlled, preferably in two degrees of freedom. It has been shown to be expedient when the X-ray apparatus is adapted as a medical C arm. The X-ray image can be one or more X-ray images.

The X-ray images, in particular the first or second X-ray image, of the alignment sight are usually prepared with a line of sight or imaging direction such that the target axis or a target axis segment of the target axis to be displayed with the first indicator structure, and the alignment sight in the X-ray image are arranged one behind the other along the line of sight in order to represent a reference point for the path of the target axis, in particular its path in the bone, with the alignment sight. An alignment of the line of sight, wherein the line of sight has a tilt angle orthogonal in relation to the first alignment plane, or does not coincide with the target line of sight, is also referred to as a rough alignment. The orthogonal tilting of the line of sight or the imaging direction of the first X-ray image from the first alignment plane indicates a tilting of the line of sight or imaging direction orthogonal to the first alignment plane in relation to the first alignment plane. This applies analogously to an orthogonal tilting of the line of sight in relation to the second alignment plane described below.

It is advantageous if the first indicator structure and/or the second indicator structure is/are formed with at least one elevation and/or at least one depression in order to form a structure identifiable in an X-ray image, in particular respectively identifiable structural features. More than one elevation or depression can also be provided expediently. The elevations or depression, which can, for example, be introduced into the alignment sight base body by machining, can be part of an alignment sight base body. It is usually provided that the connection element for connecting the intramedullary nail to the curved handle and the target guide are arranged on the curved handle at a distance from one another, preferably on different ends of the curved handle. It is expedient if the connection element and the target guide are connected to a curved handle arm of the curved handle, wherein the connection element is arranged on a first end and the targeting is arranged on a second end of the curved handle that is usually opposite to the first end. It is practical that the alignment sight is adapted as part of the first end of the curved handle or in particular forms the first end of the curved handle. The connection element can be arranged, in particular releasably, on the alignment sight, in particular the alignment sight base body.

It is beneficial if the second indicator structure is formed with a plurality of at least sectionally sequentially arranged structural elements arranged parallel to the first alignment plane or in an arrangement direction aligned parallel to the target line of sight, such that the orthogonal tilting of the line of sight from the first alignment plane in the first X-ray image is represented by a relative position of the structural elements, or is recognizable from such a position. For example, by a different relative position of the structural elements in comparison to an intended relative position of the structural elements in the second X-ray image of the alignment sight along the target line of sight. The structural elements are usually spaced apart from one another in a direction orthogonal to the arrangement direction. It goes without saying that the structural elements are represented in the X-ray image by respective images representing these. The respective structural element can be formed with an elevation and/or depression or can be formed by a part of said elevation and/or depression. For example, structural elements adapted as an elevation or depression can each be at least sectionally arranged one behind the other in arrangement direction such that a defined lateral distance, or no lateral distance, is formed in the second X-ray image of the alignment sight between the structural elements or imaging features representing these, and such that the first X-ray image of the alignment sight shows a different lateral distance, or a lateral distance in the first place, for example a gap, between the structural elements, while the line of sight is tilted orthogonally from the first alignment plane. It goes without saying that a relative position and/or a distance of the structural elements in the respective X-ray image relates to the images of the structural elements. by means of which they are shown in the X-ray image.

It is advantageous if the second indicator structure, in particular the structural elements, are adapted such that they indicate a direction of the orthogonal tilting of the line of sight from the first alignment plane in the first X-ray image. Therefore, if it is discernible in the X-ray image on these or their images in which of the two directions orthogonal to the first alignment plane the orthogonal tilting is present. This allows a misalignment of the line of sight to be detected and corrected efficiently.

This can be implemented expediently when the second indicator structure comprises at least two first structural elements and at least one second structural element, which second structural element is spaced apart from the first structural elements in an arrangement direction parallel to the first alignment plane or parallel to the target line of sight such that—when compared to the second X-ray image of the alignment sight—the first X-ray image of the alignment sight shows a direction-dependent lateral displacement of the second structural element relative to the first structural elements depending on the direction of the orthogonal tilting of the line of sight from the first alignment plane. The first structural elements are usually spaced apart from one another in a direction orthogonal to the arrangement direction. A plurality of first and/or a plurality of second such structural elements can be provided for improved recognizability. The first structural elements and the at least one second structural element can, for example, be formed with elevations and/or depressions, wherein the first structural elements and the second structural element are at least sectionally arranged sequentially in the arrangement direction. The structural elements can, for example, be adapted and arranged such that a gap, also referred to as a tilt indicator gap, exists between the first structural elements such that the second structural element is arranged in the second X-ray image along the target line of sight in a center of the gap, and the second structural element is in the first X-ray image-when the line of sight is orthogonally tilted from the first alignment plane-shown displaced from the first alignment plane in the direction of the first or other structural element, depending on the direction of the orthogonal tilting of the line of sight.

It is beneficial for good detectability in the X-ray image if the second indicator structure is formed with a plurality of gap delimiting walls such that the gap delimiting walls form two tilt indicator gaps spaced apart from one another in the first X-ray image of the alignment sight, wherein a direction of the orthogonal tilting can be read by comparing the gap widths of the tilt indicator gap. It is expedient when the one or the other tilt indicator gap has a greater decrease in its gap width depending on the direction of the orthogonal tilting of the line of sight from the first alignment plane. To determine the target alignment, it has proven effective when the gap delimiting walls in the second X-ray image of the alignment sight, or when a line of sight is located in the first alignment plane, the gap delimiting walls form two tilt indicator gaps spaced apart from one another with the same gap width. The respective tilt indicator gap is usually formed with at least one of the first structural elements and at least one of the second structural elements, which each form one of the gap delimiting walls.

Practical implementation is achievable if a first gap delimiting wall and a second gap delimiting wall each form a wall pair in order to form the respective tilt indicator gap in the first or second X-ray image, wherein the second gap delimiting wall of the first gap delimiting wall is at least sectionally arranged downstream in arrangement direction or in a direction parallel to the first alignment plane, and wherein, in one of the wall pairs, the first gap delimiting wall is arranged orthogonally to the first alignment plane in a first direction and, in the other wall pair, the first gap delimiting wall is arranged downstream of the second gap delimiting wall in a direction opposite to the first direction such that the one or the other tilt indicator gap has a greater decrease in its gap width, depending on the direction of the orthogonal tilting of the line of sight from the first alignment plane in the X-ray image of the alignment sight. The respective first gap delimiting wall and the second gap delimiting wall are usually arranged at a distance from one another in a direction orthogonal to the arrangement direction and can at least partially overlap each other in this direction. In arrangement direction, the second gap delimiting walls are preferably arranged between the first gap delimiting walls. It has proven effective if the second gap delimiting wall of a respective wall pair has a larger longitudinal extension in the arrangement direction than the first gap delimiting wall. The gap delimiting walls are preferably adapted as rails with a respective longitudinal axis substantially parallel to the arrangement direction. The respective first gap delimiting wall usually represents one of the first structural elements and the respective second gap delimiting wall represents one of the second structural elements. The respective first and second gap delimiting wall can be adapted to overlap or to be spaced apart from one another in a direction orthogonal to the arrangement direction. Arrangement direction usually refers to a direction parallel to the first alignment plane or parallel to the target line of sight.

A high readability is achievable when the alignment sight is adapted such that the first indicator structure in the second X-ray image of the alignment sight is arranged between the two tilt indicator gaps of the second indicator structures. For a simple and quick reading, it is beneficial if the first indicator structure in the second X-ray image forms an incision, wherein the incision base of the incision preferably indicates the path of the target axis. The incision walls of the incision can be formed with structural elements of the second indicator structure. For example, with respectively one of the gap delimiting walls of the tilt indicator gap.

It is advantageous if the alignment sight has a third indicator structure adapted to display in a third X-ray image of the alignment sight, in particular the first and/or second X-ray image of the alignment sight, an orthogonal tilting of the line of sight of the third X-ray image from a second alignment plane aligned at right angles to the first alignment plane, in order to align the line of sight of the third X-ray image with the second alignment plane. A reference point for a real position of a segment of the target axis in the bone displayed with the first indicator structure can thus be defined. The third X-ray image can be the first and/or second X-ray image, as a result of which the line of sight of the first or second X-ray image of the alignment sight accordingly corresponds to the line of sight of the third X-ray image of the alignment sight. A misalignment or tilting of the respective line of sight in a direction within, or parallel to, the first alignment plane can thus be detected and improved. By adjusting the line of sight of a respective X-ray image in relation to the first and second alignment plane, the line of sight can be set in an optimized manner such that a real position of the target axis or the bone drill device can be better estimated in relation to a bone structure shown in the X-ray image. It is expedient if the target line of sight also lies in the second alignment plane. The target line of sight is then defined by an intersection line of the first alignment plane and the second alignment plane, along which these intersect. It is particularly advantageous if the second alignment plane is aligned orthogonally to the target axis. If the target line of sight as a result extends orthogonally to the target axis, or the line of sight is adjustable correspondingly, longitudinal distances in an X-ray image of the alignment sight along the target line of sight correspond to longitudinal distances along the target axis.

The third indicator structure is preferably formed with a plurality of alignment structures arranged one behind the other in arrangement direction, so that a defined alignment or superimposition of the alignment structures indicates an arrangement of the line of sight of the third X-ray image within the second alignment plane. It is expedient if the third indicator structure is formed by at least one or more elevations and/or at least one or more depressions in order to form a structure that can be identified in the X-ray image. The elevations or depressions can be part of the alignment sight base body or can be introduced into the latter by machining. The first, second, and third indicator structure can be formed by common elevations and/or depressions. An embodiment of the third indicator structure or the alignment structures can be implemented in an analogous manner to the first or second indicator structure or its structural elements.

It is advantageous if the alignment structures are adapted such that they form a predetermined target superimposition contour in the third X-ray image of the alignment sight with the line of sight of the third X-ray image lying in the second alignment plane, wherein each of the alignment structures forms only a partial contour of the target superimposition contour. It goes without saying that it is expedient if the target superimposition contour is not formed by the second alignment plane when the line of sight is tilted orthogonally. For example, it can be provided that the target superimposition contour represents a depression or elevation having a defined shape, for example a parabolic shape. It is expedient if one of the alignment structures is formed by a structural feature, for example the aforementioned incision by which the first indicator structure is formed. It is practical if the alignment structures each form a depression or elevation in the X-ray image.

The bone implant, in particular the intramedullary nail, can usually be releasably connected to the curved handle, in particular in an interlocking and/or friction-locking manner, using the connection element of the curved handle. For this purpose, the connection element can comprise a first coupling element that can be releasably connected to a second coupling element of the intramedullary nail that corresponds to the shape of the first coupling element, for example by mating into one another by forming an interlocking and/or friction-locking connection. The curved handle can generally be connected to the intramedullary nail in a rotationally fixed manner. It is usually provided that when the bone implant, in particular an intramedullary nail, is connected to the connection element, a user, usually a surgeon, guides, in particular introduces, the bone implant toward or into the bone by guiding the curved handle, and aligns the bone implant in relation to the bone.

The alignment sight is usually formed from a radiopaque material. [sic: As a result, it represents a recognizable structure in an X-ray image of the latter, in particular a structure with a greater contrast in comparison with bone and/or tissue.] It can be beneficial to form other parts of the positioning device, such as a curved handle segment or the curved handle arm of the curved handle. on which the target guide is arranged, from an X-ray-transparent material such that this represents a lower-contrast structure in the X-ray image compared to the alignment sight or compared to bone and/or tissue.

It is advantageous if a positioning system is provided for positioning and/or securing an intramedullary nail in a bone, in particular a proximal femur, to treat a fracture, in particular a fracture of a proximal femur, wherein the positioning system has a positioning device and an intramedullary nail that can be introduced into an intramedullary canal of the bone, wherein the intramedullary nail has a bore projecting transversely in relation to a longitudinal axis of the intramedullary nail to accommodate a coupling device, in particular a bone screw, to introduce the coupling device into the bore such that the coupling device projects beyond the bore on at least one side, in particular on both sides, wherein the coupling device can be secured on the end face in a bone part, in particular a femoral head, for example with a thread. The positioning device can in this case be expediently adapted as described, in particular above. The bone drill device can be adapted as a bone drill or guide wire, in particular a K-wire. It is advantageous if the bone drill device and/or the coupling device is a component of the positioning system. The intramedullary nail can usually be releasably connected, in particular as stated above, to the connection element of the positioning device in order to introduce the intramedullary nail into the intramedullary canal of the bone using the positioning device. The intramedullary nail is usually adapted such that the bore axis of the bore aligns with the target axis when the intramedullary nail is connected to the connection element. The target axis usually defines the path along which the bone screw is to be introduced into the intramedullary nail.

By preparing an X-ray image of the alignment sight, in particular according to the aforementioned second X-ray image, an orthogonal tilting of the line of sight from the alignment plane is discernible, or is indicated, by the second indicator structure on the X-ray image. The line of sight of the X-ray image and the first alignment plane can thus be aligned with one another such that. when an intramedullary nail is connected, in particular releasably, to the connection element, a bone drill device, such as a target wire, in particular a K-wire, can be guided along the target axis with the target guide of the positioning device, in particular through the bore, and wherein said bone drill device has a path indicated with the first indicator structure in the X-ray image.

It is practical if the target guide comprises a guide device and a drill device guide, wherein the drill device guide comprises a drill device canal for guiding the bone drill device, wherein the drill device guide has an interlocking, in particular releasable, connection to the guide device such that the drill device guide can be displaceably guided along the target axis relative to the guide device, to guide the drill device canal to a bone surface of the bone or to the intramedullary nail when an intramedullary nail is introduced into the bone. The bone drill device is thus protected by the drill device guide. A diameter of the drill device canal is usually adapted according to a diameter of the bone drill device. Depending on the bone drill device to be used, different drill device guides with diameters of the drill device canal adapted to a diameter of the bone drill device can be used expediently. In a state of the drill device guide of the drill device canal connected to the guide device, the drill device canal is typically arranged along a path of the longitudinal axis of the drill device guide, or a longitudinal axis of the drill device canal is aligned along the target axis in order to guide the drill device along the target axis. The drill device canal can, for example, be adapted as a drill hole. The drill device guide generally comprises a rod-shaped segment, which—in a state guided by the guide device—forms an end of the drill device guide facing the intramedullary nail.

or is in particular substantially rod-shaped. The guide device and the drill device guide are usually adapted such that the drill device guide—when in a deployed position projected in the direction of the intramedullary nail—bridges a path section between the guide device and the intramedullary nail in order to guide the bone drill device in a protected manner along said path section. It is expedient if the drill device guide can be substantially guided up to the intramedullary nail, wherein a remaining distance to the intramedullary nail can vary depending on a thickness of the respective bone. It is usually provided that the drill device guide is pushed through the body tissue surrounding the bones up to the proximity of the bone surface of the bone or to the proximity of the intramedullary nail. The bone drill device can thus be protected by the drill device guide while being positioned in the drill device canal up to the proximity of the intramedullary nail or bone in order to then insert the bone drill device further into the bone. The guide device can be formed with a guide canal into which the drill device guide can be introduced in an interlocking manner. The guide canal generally defines a guide canal axis matching the target axis, along which the drill device guide can be displaceably guided.

It is advantageous if the target guide comprises a tissue protection jacket that can be moved relative to the guide device in order to at least partially envelop the drill device guide in a deployed position of the drill device guide, in which the drill device guide is guided by the guide device towards the bone or intramedullary nail in order to separate the drill device guide from a body part tissue surrounding the bone. This makes it possible to reduce, in particular prevent, any impairment of the body part tissue when the drill device guide is moved. The tissue protection jacket can expediently be formed at least segmentally with an envelope surface, which, when the drill device guide is introduced into the drill device guide canal, envelops the drill device guide at least partially, preferably to a large extent, in particular completely, in the circumferential direction of the drill device guide. This applies in particular to an end element of the tissue protection jacket facing the intramedullary nail. Advantageously, the tissue protection jacket is adapted to envelop the drill device guide in its deployed position substantially along a large part, in particular along an entire lengthwise extension of the drill device guide between the deployed position and the guide device. A high degree of practicability is achievable when the tissue protection jacket can be connected to the guide device in an interlocking, in particular releasable, manner such that the tissue protection jacket can be displaced along the target axis relative to the guide device in order to guide the tissue protection jacket toward a bone surface of the bone or the intramedullary nail when the intramedullary nail is introduced into the bone. It is advantageously provided that the tissue protection jacket comprises a drill device guide canal for guiding the drill device guide, wherein the drill device guide can be connected to the drill device guide canal in an interlocking, in particular releasable, manner such that the drill device guide can be displaceably guided along the target axis relative to the tissue protection jacket. As a result, the tissue protection jacket can be guided through along the target axis between body part tissue up to the proximity of the bone; the drill device guide can thereafter then be guided into the deployed position. It is practical if the tissue protection jacket can be introduced into the guide canal of the guide device in an interlocking manner. The tissue protection jacket can then be introduced in an interlocking manner into the guide canal of the guide device in order to introduce the tissue protection jacket up to the vicinity of the intramedullary nail or bone; the drill device guide can thereafter then be introduced in an interlocking manner into the drill device guide of the tissue protection jacket in order to introduce the drill device guide up to the proximity of the intramedullary nail while at least partially enveloped by the tissue protection jacket.

It is advantageous if the guide device comprises a plurality of guide canals, wherein the guide canals are each adapted for a displaceably guided connection with a drill device guide or a tissue protection jacket, wherein preferably different guide canals have different target axes or define different guide canal axes to guide a bone drill device along these. Accordingly, it can be provided that the guide device comprises at least one or more additional guide canals, each of which defines a further guide canal axis or further target axis in order to connect, in particular releasably, a drill device guide and/or a tissue protection jacket displaceably guided along the further guide canal axis, in particular in the aforementioned manner to the guide device. A drill device, such as a bone drill or a guide wire, in particular a K-wire, can then in the aforementioned manner be guided with the drill device guide along the further guide canal axis. The intramedullary nail can expediently comprise at least one or more further bores, wherein a respective bore axis of the further bores respectively coincides with one of the guide canal axes. It can be provided that a further coupling element, such as a bone screw, is introduced into the respective further bore, which bone screw projects beyond the respective further bore at least on one side or on both sides in order to secure the intramedullary nail in the bone.

It is expedient when the target guide, in particular the guide device, is releasably connected to the curved handle arm of the curved handle in order to release the target guide or guide device from the curved handle arm of the curved handle. A connection device can expediently be provided, with which the target guide, in particular the guide device, and the curved handle arm can be releasably connected to one another, in particular in an interlocking and/or friction-locking manner. This makes it possible for the target guide, in particular the guide device, to be connected to the curved handle only after the intramedullary nail was introduced into the bone. This simplifies handling during the procedure. It is practical when the alignment sight is releasably connected to the curved handle arm with a connection adapter, in particular in an interlocking and/or friction-locking manner.

The further object is achieved by a method of the aforementioned type when a first X-ray image of the alignment sight of the curved handle is prepared along a line of sight, whereupon an adjustment of the line of sight to the first alignment plane is carried out on the basis of a second indicator structure of the alignment sight, which indicator structure is adapted to indicate an orthogonal tilting of the line of sight from the first alignment plane in an X-ray image of the alignment sight. It is usually provided that a positioning device as described in this document, or a described positioning system, is used to carry out the method. As stated, an adjustment or alignment of the target axis, the first indicator structure, and the line of sight of the X-ray image can be carried out simply and practically so that a time requirement and/or a radiation exposure can be reduced.

The target axis is usually aligned transversely to a length axis of the intramedullary nail, preferably such that it parts the intramedullary nail or coincides with a bore axis of a bore of the intramedullary nail, as in particular stated above. In order to treat a proximal femur, the target axis is generally aligned with the longitudinal axis of the intramedullary nail at an angle at which a longitudinal axis of the femur is aligned in relation to a longitudinal axis of the femur neck, usually at an angle of 110° and 140°. If the bone is a femur, the target line of sight of the X-ray usually corresponds to a lateral-medial view of the femur or body whose part is the femur.

It is expedient if a bone drill device, in particular a K-wire, is guided into the bone along the target axis via the target guide of the curved handle, wherein the line of sight is adjusted on the basis of the second indicator structure before and/or after the bone drill device is visible in the first X-ray image for a comparison with the first indicator structure. An optimized adjustment of the line of sight is achievable before and/or after the bone drill device is visible in the X-ray image.

It is usually provided that the intramedullary nail comprises a bore extending transversely in relation to a longitudinal axis of the intramedullary nail for accommodating a coupling device, in particular a bone screw, wherein the bone drill device, in particular the K-wire, is guided through the bore along the target axis via the target guide. The bore typically comprises a bore axis that corresponds to the target axis. The bone drill device can be a bone drill or a targeting wire, in particular a K-wire. Using the coupling device, the intramedullary nail can be secured in the bone, or a bone material of the bone can be coupled to the intramedullary nail using the coupling device. For this purpose, the coupling device can be introduced into the bore of the intramedullary nail such that the coupling device projects beyond the bore at least on one side, in particular on both sides. The coupling device is usually anchored in the bone material of the bone with at least one end of the coupling device, usually with the leading end in insertion direction of the coupling direction into the bone. For this purpose, the end can have a thread.

As a rule, a bone bore canal is introduced into the bone along the target axis with the bone drill device, usually through the bore of the intramedullary nail: thereafter, the coupling device is then introduced into the bore along the bone bore canal. If the bone is a proximal femur, the bone bore canal generally extends along the target axis up to the femoral head, wherein the coupling device is preferably introduced into the bore such that one end of the coupling device projects into, or is anchored in, the femoral head, for example with a thread.

It is beneficial if a bone drill device adapted as a targeting wire, in particular a K-wire, having a diameter smaller than the coupling device, is initially introduced into the bone along the target axis, in particular in the aforementioned manner, according to which a bone bore canal is introduced into the bone with a bone drill, wherein the targeting wire acts as a guide. The coupling device can then be introduced into the bore via the bone bore canal, usually such that the coupling device projects beyond the bore at least on one side or on both sides. The second bone drill device or the bone drill and/or the coupling device are in this case generally adapted to be cannulated in order to guide them along the targeting wire. For this purpose, the bone drill device or the coupling device can comprise a feed-through canal. If the bone is a proximal femur, the coupling device is usually secured in the femoral head with an end of the coupling device. The guide wire is usually removed after the coupling device is introduced into the bore of the intramedullary nail and in particular into the femoral head.

It can be advantageous to introduce a further guide wire, in particular a K-wire, at an axis offset into the femoral head in order to avoid a rotation of the femoral head when drilling the bone bore canal, in particular the second bone bore canal, and/or when introducing the coupling device into the bore of the intramedullary nail, an expanded guide wire can extend into the femoral head along a further target axis, preferably through a further bore of the intramedullary nail. The further guide wire can expediently be introduced into the femoral head with the guide device, for example via a further guide canal of the guide device.

The first X-ray image of the alignment sight usually refers to an X-ray image of the alignment sight along a line of sight, while orthogonally tilting the line of sight from the first alignment plane. The second X-ray image of the alignment sight usually refers to an X-ray image of the alignment sight along a line of sight located in the first alignment plane.

Further features, advantages, and effects are shown in the exemplary embodiments shown below. The drawings to which reference is made show:

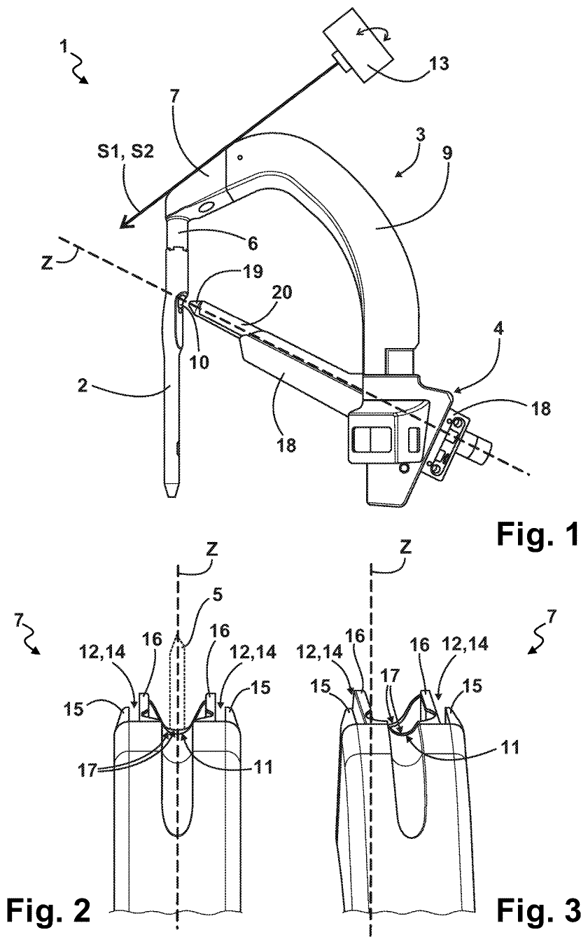

FIG. 1 a positioning device for an intramedullary nail;

FIG. 2 and FIG. 3 an alignment sight of a positioning device from different lines of sight;

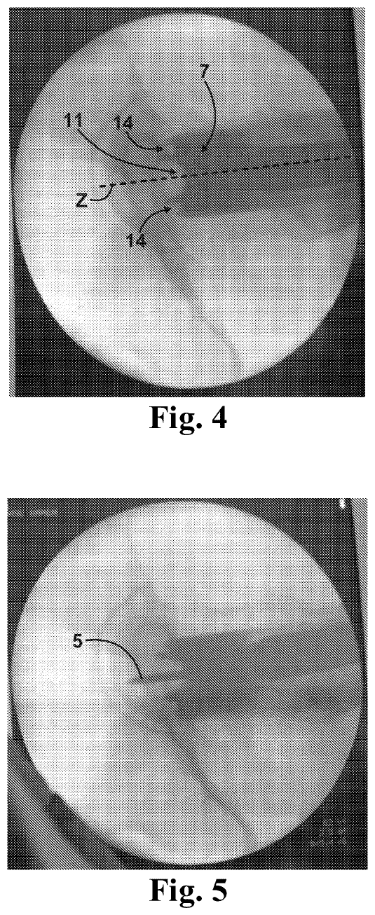

FIG. 4 an X-ray image of a proximal femur along a line of sight that lies in a plane with a target axis and a first indicator structure;

FIG. 5 a further X-ray image according to FIG. 4 with a K-wire introduced into the bone along the target axis;

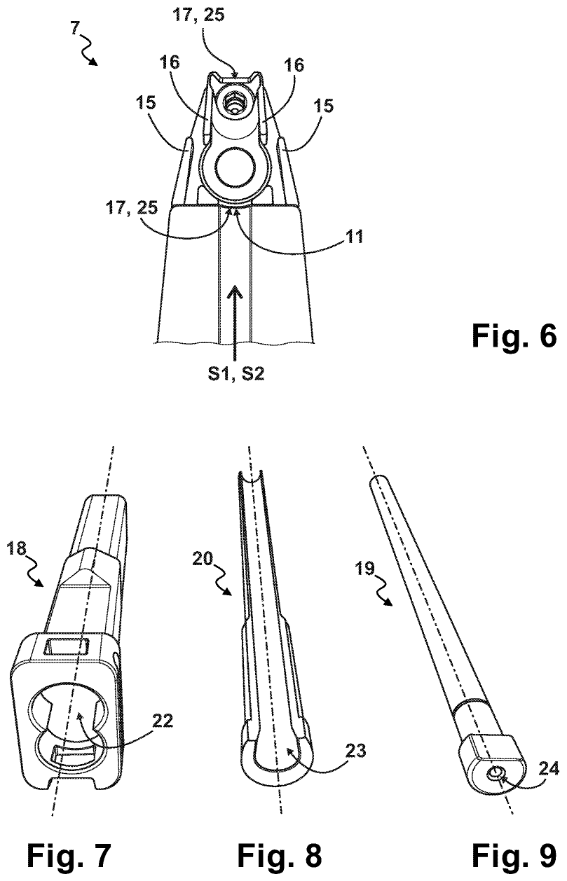

FIG. 6 the alignment sight of FIG. 2 and FIG. 3 in an oblique plan view onto indicator structures of the alignment sight;

FIG. 7 a guide device of a target guide;

FIG. 8 a tissue protection jacket of a target guide;

FIG. 9 a drill device guide of a target guide.

FIG. 1 shows a schematic illustration of a positioning device 1 for an intramedullary nail 2 for positioning and securing the intramedullary nail 2 in an intramedullary canal of a bone, in particular a proximal femur. The purpose is usually to treat a fracture of the bone, often a femoral neck fracture in the case of a proximal femur. The positioning device comprises a curved handle 3 with a target guide 4 for guiding a bone drill device 5, for example a K-wire, along a target axis Z, a connection element to connect the intramedullary nail 2 to the curved handle 3 in a rotationally fixed manner, and an alignment sight 7 to display a path of the target axis Z in an X-ray image to align the target axis Z when the intramedullary nail 2 is introduced in the bone with the alignment sight 7. The connection element 6 and the target guide 4 are arranged spaced apart from one another on a curved handle arm 9 of the curved handle 3, usually at different ends of the curved handle arm 9.

The intramedullary nail 2 comprises a bore 10 extending transversely to its longitudinal axis to accommodate a coupling device, such as a bone screw, in order to fasten the intramedullary nail 2 by introducing the coupling device into the bore 10 such that the coupling device projects beyond the bore 10 in the bone. The coupling device is usually anchored on the end face in a bone part, usually in the femoral head in the case of a proximal femur. When an intramedullary nail 2 is connected to the connection element 6, an axis of the bore 10 and the target axis Z coincide such that the target axis Z projects through the bore 10, as shown in FIG. 1. It is usually provided that a bone drill device 5 adapted as a K-wire is first guided along the target axis Z through the bore 10 using the target guide 4, and that a bone drill is then introduced into the bone along the target axis Z for a bore 10 of a bone bore canal, wherein the K-wire acts as a guide for the bone drill. The coupling device can then be introduced into the bore 10 of the medullary canal via the bone bore canal.

An alignment of the target axis Z in the bone in order to guide the K-wire or the coupling device into a suitable depth and position in the bone is performed while preparing X-ray images of the alignment sight 7 such that a first indicator structure 11 of the alignment sight 7 shown in the X-ray images represents a reference point for a path of the target axis Z in the bone. The X-ray images are generally performed with an X-ray apparatus 13 with a controllably variable line of sight S1 of the X-ray image. The first indicator structure 11 is adapted to display the path of the target axis Z in an X-ray image of the alignment sight 7 along a target line of sight S2 lying in a first alignment plane formed with the target axis Z and the first indicator structure 11. This is shown in FIG. 2, wherein FIG. 2 shows the first alignment plane spanned through the target axis Z at right angles to the drawing plane. In order to permit alignment of the line of sight S1 of an X-ray image of the alignment sight 7 according to the target line of sight S2, i.e., to detect a misalignment of the line of sight S1, the invention provides that the alignment sight 7 comprises a second indicator structure 12 formed to indicate in the X-ray image an orthogonal tilting of the line of sight S1 from the first alignment plane. This is shown in FIG. 2 and FIG. 3.

FIG. 2 and FIG. 3 show schematic illustrations of an alignment sight 7 of a positioning device, in particular that of FIG. 1, wherein the alignment sight 7 is shown in FIG. 2 from a vantage point of the alignment sight 7 along the first alignment plane, and in FIG. 3 from a vantage point onto the alignment sight 7 with orthogonal tilting of the first alignment plane. The first indicator structure 11 is formed with a depression such that the first indicator structure 11 image forms an incision, in particular a U-shaped or V-shaped incision, in an X-ray along the target line of sight S2 in order to display the path of the target axis Z with said incision, analogous to FIG. 2. The path of the target axis Z is in particular marked by a base of the incision. For an accurate display, it is expedient when the depression or the incision have a tapered cross-section along their depth direction. The indicator structures are preferably formed as elevations or depressions.

The second indicator structure 12 is formed with a plurality of gap delimiting walls spaced apart from one another and embodied in particular as rails that are substantially parallel, and orthogonal, to the length axis of the first alignment plane or parallel to the line of sight S1, such that the gap delimiting walls in an X-ray image of the alignment sight 7 along the target line of sight S2 form two tilt indicator gaps 14 spaced apart from one another, wherein a first gap delimiting wall 15 and a second gap delimiting wall 16 each form a wall pair to form the respective tilt indicator gap 14, wherein each second gap delimiting wall 16 is at least sectionally arranged downstream of the first gap delimiting wall 15 in a direction parallel to the first alignment plane, and wherein, in one of the wall pairs, the first gap delimiting wall 15 is arranged downstream of the second gap delimiting wall 16 in a first direction orthogonal to the first alignment plane and, in the other wall pair, the first gap delimiting wall 15 is arranged downstream of the second gap delimiting wall 16 in a direction opposite to the first direction, such that the one or the other tilt indicator gap 14 has a greater decrease in its gap width depending on the direction of the orthogonal tilting of the line of sight S1 from the first alignment plane in the X-ray image of the alignment sight 7. An arrangement of the gap delimiting walls is in particular evident from FIG. 2. FIG. 3, and FIG. 6, wherein FIG. 6 shows a schematic illustration of the alignment sight in an oblique plan view onto the gap delimiting walls. Due to this arrangement of the gap delimiting walls, a direction of the orthogonal tilting of the line of sight S1 from the target line of sight S2 is efficiently and easily readable in the X-ray image. It is advantageous if the tilt indicator gap 14 has equally large gap widths in an X-ray image along the target line of sight S2. A particularly sensitive display of the orthogonal tilting can be achieved by arranging the first indicator structure 11 between the tilt indicator gaps 14. As can be seen analogously in FIG. 2 and FIG. 3, the tilt indicator gap 14 in FIG. 2 has equally large gap widths, while in FIG. 3, one of the tilt indicator gaps 14 has a gap width smaller than the other tilt indicator gap 14 due to the orthogonal tilting, depending on a respective direction of the orthogonal tilting of the first alignment plane.

It can be practical if the alignment sight 7 has a third indicator structure 17 in order to display in an X-ray image of the alignment sight 7 an orthogonal tilting of the line of sight S1 from a second alignment plane aligned at right angles to the first alignment plane defined by the target axis Z and the first indicator structure 11. As a result, the line of sight S1 can also be practically aligned with respect to the second alignment plane. The third indicator structure 17 can preferably be formed with two alignment structures 25 adapted as depressions and arranged one behind the other in a direction parallel to the first alignment plane, wherein a defined superimposition of the depressions in the X-ray image forms a target superimposition contour. As can be seen in FIG. 2 and FIG. 3, this can be implemented by one of the depressions of the third indicator structure 17 being implemented by the depression of the first indicator structure 11 and a further depression being arranged behind the former in a direction of the target line of sight S2.

FIG. 4 shows an X-ray image of an alignment sight 7 according to FIG. 2 or FIG. 3 as part of an exemplary treatment of a fracture of a proximal femur. In particular, the alignment sight 7 is part of a positioning device 1 according to an embodiment according to FIG. 1. The X-ray image shows an alignment of the target axis Z onto a femoral head in order to introduce a K-wire along the target axis Z into the femoral head after the target axis Z is aligned. For illustration purposes, the target axis Z is drawn as a dashed line in the X-ray image. The line of sight S1 of the X-ray image is aligned relative to the first alignment plane such that the tilt indicator gap 14 has substantially equal gap widths: the incision of the first indicator structure 11 thus indicates the path of the target axis Z. FIG. 5 shows a further X-ray image of the alignment sight 7 according to FIG. 4, wherein a K-wire is introduced into the femoral head along the target axis Z. The longitudinal axis of the K-wire extends according to the path indicated with the incision of the first indicator structure 11.

FIG. 6 shows a schematic illustration of the alignment sight 7 of FIG. 2 and FIG. 3 in an oblique plan view onto the first indicator structure 11 and the second indicator structure 12 of the alignment sight 7. Respectively one of the first gap delimiting walls 15 and one of the second gap delimiting walls 16, as explained above, form one of the wall pairs in order to form one of the tilt indicator gaps 14 in the X-ray image. The first gap delimiting wall 15 and the second gap delimiting wall 16 of the respective wall pair are spaced apart from one another in a direction orthogonal to the target line of sight S2 and can also overlap one another. The depression of the first indicator structure 11 is located between the two wall pairs. The centrally located circular structures shown in FIG. 6 represent boreholes in the alignment sight body, which were incorporated while producing the alignment sight 7 in order to implement the indicator structures into an alignment sight body of the alignment sight 7.

In order to guide the bone drill device 5 with the target guide 4, it is expedient if the target guide 4 comprises a guide device 18 and a drill device guide 19, wherein the guide device 18 is adapted to guide the drill device guide 19, and the drill device guide 19 is adapted to guide the bone drill device 5, shown in FIG. 1. The guide device 18 is usually releasably connected to the curved handle arm 9. The drill device guide 19 can preferably be arranged releasably on the guide device 18. The drill device guide 19 can be stabilized with the drill device guide 19 in order to guide the bone drill device 5 toward the bone. The drill device guide 19 is usually guided toward the bone through and between body part tissue surrounding the bone. In order to separate the drill device guide 19 and body part tissue in order to protect the body part tissue, it is expedient if the target guide 4 comprises a tissue protection jacket 20 that at least partially envelops the drill device guide 19. FIG. 7 shows a schematic illustration of a guide device 18. FIG. 8 shows a schematic illustration of a tissue protection jacket 20, and FIG. 9 shows a schematic illustration of a drill device guide 19 of a target guide 4, in particular that of FIG. 1. The guide device 18 comprises a guide canal 22 in order to introduce the drill device guide 19 and preferably the tissue protection jacket 20 in an interlocking manner displaceably along the guide canal 22 into the guide canal 22. The invention provides that the guide canal 22 is aligned with the curved handle 3 such that a guide canal axis of the guide canal 22 coincides with the target axis Z: the drill device guide 19 or the tissue protection jacket 20 can thus be displaced along the target axis Z, as can be seen in FIG. 1. The drill device guide 19, which is usually substantially rod-shaped, comprises a drill device canal 24 through which a bone drill device 5, in particular a K-wire, can be guided. The tissue protection jacket 20 comprises a drill device guide canal 23 in order to introduce the drill device guide 19 into the drill device guide canal 23 in an interlocking manner displaceably along the drill device guide canal 23. The tissue protection jacket 20 is adapted to at least partially envelop the drill device guide 19. The invention expediently provides that the tissue protection jacket 20 can be introduced into the guide canal 22 such that the tissue protection jacket 20 can be deployed guided by the guide canal 22 relative to the guide canal 22 in the direction of the intramedullary nail 2 in order to guide the tissue protection jacket 20 toward the intramedullary nail 2 or bone, this is shown in FIG. 1. The drill device guide 19 can then be guided along the drill device guide canal 23 toward the intramedullary nail 2 or bone. The invention in this case provides that a longitudinal axis of the drill device canal 24 coincides with the target axis Z. A drill device, such as a K-wire, can then be introduced into the bone through the drill device canal 24 of the drill device guide 19 guided along the target axis Z. If a tissue protection jacket 20 is not used, the drill device guide 19 can be introduced into the guide canal 22 such that the drill device guide 19 can be deployed guided by the guide canal 22 relative to the guide canal 22 in the direction of the intramedullary nail 2 in order to guide the drill device canal 24 toward the intramedullary nail 2. For this purpose, a cross-section of the drill device guide 19 can be adapted to correspond to the shape of a cross-section of the guide canal 22

It is therefore advantageous if the positioning device 1 comprises an alignment sight 7 with a first indicator structure 11 for displaying a path of the target axis Z in an X-ray image of the alignment sight 7 along a target line of sight S2, wherein the first indicator structure 11 and the target axis Z define a first alignment plane, and wherein the alignment sight 7 comprises a second indicator structure 12 adapted to indicate an orthogonal tilting of the line of sight S1 from the first alignment plane along a line of sight S1 in an X-ray image of the alignment sight 7. As a result, the line of sight S1 of the X-ray image of the alignment sight 7 can be aligned according to the first alignment plane or target line of sight S2. When an intramedullary nail 2 is introduced into the intramedullary canal of a bone with the positioning device, the first indicator structure shown in an X-ray image of the alignment sight 7 with a line of sight S1 adapted to the target line of sight S2 represents a reference point for a path of the target axis Z in the bone. If the second indicator structure 12 is formed with two tilt indicator gaps 14, which are preferably embodied with elevations or depressions, wherein a direction of the orthogonal tilting from the first alignment plane can be read on the tilt indicator gaps 14, a compact structure can be realized and a simple and efficient determination of an orthogonal tilting or alignment of the line of sight S1 of an X-ray image can be performed.

1. A positioning device for a bone implant, in particular an intramedullary nail, for positioning and/or securing the bone implant, usually for treating a bone fracture, in particular a fracture of a proximal femur, wherein the positioning device has a curved handle with a target guide, such as a target aperture, for guiding a bone drill device, wherein the target guide defines a target axis, and wherein the curved handle comprises a connection element to releasably connect the bone implant to the curved handle, and an alignment sight, wherein the alignment sight comprises a first indicator structure in order to display a path of the target axis with the first indicator structure in a second X-ray image of the alignment sight along a target line of sight located in a first alignment plane formed by the target axis and the first indicator structure, wherein the alignment sight comprises a second indicator structure adapted to indicate an orthogonal tilting of the line of sight from the first alignment plane along a line of sight deviating from the target line of sight in a first X-ray image of the alignment sight, to permit an adjustment of the line of sight to the target line of sight, wherein the second indicator structure is formed with a plurality of gap delimiting walls such that the gap delimiting walls form two tilt indicator gaps spaced apart from one another in the first X-ray image of the alignment sight, wherein a direction of the orthogonal tilting is read by comparing the gap widths of the tilt indicator gaps.

2. The positioning device according to claim 1, wherein the first indicator structure and/or the second indicator structure is formed with at least one elevation and/or at least one depression in order to form a structure that can be identified in an X-ray image.

3.-4. (canceled)

5. The positioning device according to claim 1, wherein the respective tilt indicator gap is formed in the X-ray image by a first gap delimiting wall and a second gap delimiting wall each that form a wall pair, wherein the second gap delimiting wall is at least sectionally arranged downstream of the first gap delimiting wall in a direction parallel to the first alignment plane, and wherein, in one of the wall pairs, the first gap delimiting wall is arranged downstream of the second wall limiting wall in a first direction orthogonal to the first alignment plane and in a second direction opposite to the first direction in the other wall pair such that the one or the other tilt indicator gap has a greater decrease of its gap width, depending on the direction of the orthogonal tilting of the line of sight from the first alignment plane in the X-ray image of the alignment sight.

6. The positioning device according to claim 1, wherein the alignment sight is adapted such that the first indicator structure in the second X-ray image is arranged between the two tilt indicator gaps, wherein the first indicator structure is preferably implemented with an incision.

7. The positioning device according to claim 1, wherein the alignment sight comprises a third indicator structure adapted to indicate in a third X-ray image of the alignment sight, in particular of the first and/or second X-ray image of the alignment sight, an orthogonal tilting of the line of sight of the third X-ray image from a second alignment plane, which is aligned at right angles to the first alignment plane, in order to align the line of sight of the third X-ray image with the second alignment plane.

8. The positioning device according to claim 7, wherein the third indicator structure is formed with several alignment structures arranged one behind the other in a direction parallel to the target line of sight such that a defined alignment or superimposition of the alignment structures indicates an arrangement of the line of sight of the third X-ray image within the second alignment plane.

9. A positioning system for positioning and/or securing an intramedullary nail in a bone, in particular a proximal femur, to treat a fracture, in particular a fracture of a proximal femur, wherein the positioning system has a positioning device and an intramedullary nail that can be introduced into an intramedullary canal of the bone, wherein the intramedullary nail has a bore projecting transversely in relation to a longitudinal axis of the intramedullary nail to accommodate a coupling device, in particular a bone screw, to introduce the coupling device into the bore such that the coupling device projects beyond the bore on at least one side, in particular on both sides, wherein the coupling device can be secured on the end face in a bone part, in particular a femoral head, wherein the positioning device is adapted according to claim 1.

10. The positioning system according to claim 9, wherein the target guide comprises a guide device and a drill device guide, wherein the drill device guide comprises a drill device canal for guiding the bone drill device, wherein the drill device guide can be connected, in particular releasably, to the guide device in an interlocking manner such that the drill device guide can be guided displaceably along the target axis relative to the guide device, to guide the drill device canal to a bone surface of the bone or to the intramedullary nail when the intramedullary nail is introduced into the bone.

11. The positioning system according to claim 10, wherein the target guide comprises a tissue protection jacket that can be moved relative to the guide device in order to at least partially envelop the drill device guide in a deployed position, wherein the drill device guide is guided in the direction of bone or intramedullary nail in order to separate the drill device guide from a body part tissue surrounding the bone.

12. A method for treating a bone fracture, in particular a proximal femoral fracture, wherein an intramedullary nail is introduced into an intramedullary canal of a bone, in particular a proximal femur, using a positioning device, in particular a positioning device according to claim 1, wherein the intramedullary nail is connected, in particular releasably, to a curved handle of the positioning device, wherein the curved handle comprises a target guide for guiding a bone drill device into the bone along a target axis, wherein the curved handle has an alignment sight for aligning the target axis, wherein the alignment sight comprises a first indicator structure adapted to indicate a path of the target axis in a second X-ray image of the alignment sight along a target line of sight located in a first alignment plane formed by the target axis and the first indicator structure, wherein a first X-ray image of the alignment sight of the curved handle is prepared along a line of sight, according to which the line of sight is adjusted to the first alignment plane based on a second indicator structure of the alignment sight adapted to indicate an orthogonal tilting of the line of sight from the first alignment plane in an X-ray image of the alignment sight, wherein the second indicator structure is formed with a plurality of gap delimiting walls such that the gap delimiting walls form two tilt indicator gaps spaced apart from one another in the first X-ray image of the alignment sight, wherein a direction of the orthogonal tilting is read by comparing the gap widths of the tilt indicator gaps.

13. The method according to claim 12, wherein a bone drill device, in particular a K-wire, is guided along the target axis into the bone using the target guide of the curved handle, wherein the line of sight is adjusted on the basis of the second indicator structure before and/or after the bone drill device is visible in the first X-ray image for a comparison with the first indicator structure.

14. The method according to claim 12, wherein the intramedullary nail comprises a bore extending transversely in relation to a longitudinal axis of the intramedullary nail to accommodate a coupling device, in particular a bone screw, wherein the bone drill device, in particular the K-wire, is guided with the target guide through the bore along the target axis.

15. The method according to claim 12, wherein a bone drill device adapted as a targeting wire, in particular a K-wire, which bone drill device has a diameter smaller than the coupling device, is guided along the target axis through the bore in a first step, after which a bone bore canal is introduced into the bone along the target axis with a bone drill, wherein the target wire acts as a guide, after which the coupling device is introduced into the bore via the bone bore canal.
